(19) United States
(12) Patent Application Publication
(10) Pub. No.: US 2023/0131184 A1
(43) Pub. Date: Apr. 27, 2023

(54) INTERMITTENT WARMING OF A BIOLOGIC SAMPLE INCLUDING A NUCLEIC ACID

(71) Applicant: HEWLETT-PACKARD DEVELOPMENT COMPANY, L.P., Spring, TX (US)

(72) Inventors: Carson DENISON, Corvallis, OR (US); Erik TORNIAINEN, Corvallis, OR (US); Richard SEAVER, Corvallis, OR (US)

(73) Assignee: HEWLETT-PACKARD DEVELOPMENT COMPANY, L.P., Spring, TX (US)

(21) Appl. No.: 17/912,189

(22) PCT Filed: Mar. 30, 2020
(86) PCT No.: PCT/US2020/025666

§ 371 Date: Sep. 16, 2022

(51) Int. Cl. B01L 7/00; C12Q 1/686

(52) U.S. Cl. CPC B01L 7/525; C12Q 1/686; B01L 2200/147; B01L 2300/087; B01L 2300/088; B01L 2300/1805; B01L 2300/185

(57) ABSTRACT

Intermittent warming of a biologic sample including a nucleic acid includes receiving at a first end of a channel of a microfluidic device, a biologic sample including a nucleic acid, and warming a subset of a plurality of heating elements disposed adjacent to the channel. The method includes warming the heating elements to a particular temperature of a particular warming and cooling protocol. The method includes moving the biologic sample from the first end of the channel to a second end of the channel opposite the first end at a particular flow rate associated with the warming and cooling protocol, and intermittently warming the biologic sample using the subset of heating elements while the biologic sample moves from the first end of the channel to the second end of the channel.

### BACKGROUND

Polymerase chain reaction (PCR) is a method used in molecular biology to make many copies of a nucleic acid segment. Using PCR, a single copy (or more) of a nucleic acid sequence is exponentially amplified to generate thousands to millions or more copies of that particular nucleic acid segment. Many PCR methods rely on thermal cycling. Thermal cycling exposes reactants to repeated cycles of warming and cooling to permit different temperature-dependent reactions to occur.

PCR is a temperature-mediated process including cycling a biologic sample between set temperatures. Single-strand nucleic acids are used so that two primer sequences may bind upstream and downstream of the region to be amplified. To allow this to occur, the first step of PCR is denaturation or separation of the two strands at around 94-98 degrees Centigrade (C). Primer annealing occurs around 45-55 degrees C. and allows the thermo-stable polymerase to bind to defined regions of double stranded DNA. The next stage is elongation of the double stranded copy where the temperature is raised to around 72 degrees C. for the enzyme catalysis to proceed. Finally, temperature is returned to 94 degrees C. for denaturation to single-stranded DNA that allows the cycle to repeat. A PCR reaction may take up to a few hours to complete depending on the quality and concentration of the target nucleic acid. Faster PCR cycle times may increase the workflow efficiency in PCR-based technologies.

### BRIEF DESCRIPTION OF THE DRAWINGS

FIG. 1 illustrates an example method for intermittent warming of a biologic sample including a nucleic acid, consistent with the present disclosure.

FIG. 2 illustrates an example apparatus including a plurality of heating elements, consistent with the present disclosure.

FIG. 3 illustrates an example apparatus including narrowed adiabatic zones, consistent with the present disclosure.

FIG. 4 illustrates an example apparatus including liquid cooling elements, consistent with the present disclosure.

FIG. 5 illustrates an example apparatus including multiple viewing windows, consistent with the present disclosure.

FIG. 6 illustrates an example apparatus including a multiplexed fluidic channel, consistent with the present disclosure.

FIG. 7 illustrates an example apparatus including a non-straight fluidic channel, consistent with the present disclosure.

FIG. 8 illustrates an example apparatus including a looped fluidic channel, consistent with the present disclosure.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

In many methods, PCR is carried out in a volume of 10-200 microliters (μL) in a thermal cycler. The thermal cycler refers to or includes an apparatus to amplify segments of nucleic acids via the PCR process. As the separate steps of the PCR process can be carried out at different temperatures, one or several warming steps may be performed, and where applicable, cooling steps may be performed during or between the steps of the PCR in which the reaction volume or parts thereof are cooled.

The thermocycling of a PCR process can be dependent upon several factors. The additional time of PCR is a function of the time to reach the desired temperatures for the temperature-mediated processes (e.g., denaturation, primer annealing, and elongation/synthesis). Shortening warming and cooling times results in more rapid transition and shorter cycling times.

In many PCR systems, once a thermal cycler is built, the design is locked into a specific family of thermal protocols. Changing the flow velocity can speed up or slow down the amount of time the fluid spends at each temperature, but it does so for all phases of the reaction equally. In other words, such systems do not allow for thermal protocols with variable ratios of time spent at denature, anneal, and extension (e.g., topologically different thermal protocols) to be used within the same PCR device. As used herein, a thermal protocol refers to or includes a sequence of temperatures, and a corresponding amount of time at which a biologic sample is held at a particular temperature in the sequence for amplification of nucleic acids within the biologic sample. Also as used herein, a topologically different thermal protocol refers to or includes a thermal protocol that differs in terms of where in the microfluidic device a particular temperature is applied.

Intermittent warming of a biologic sample including a nucleic acid, consistent with the present disclosure, allows for customized, and modifiable, thermal protocols implemented by a PCR device. The protocol can be changed by means of a computer which turns on and off different temperature-controlled zones, without mechanical changes to the PCR device. Moreover, many PCR devices operate on timescales of seconds. However, intermittent warming of a biologic sample including a nucleic acid, consistent with the present disclosure, allows for completion of a warming phase and/or a cooling phase of PCR within tens of milliseconds Additionally, while some PCR devices include so-called dead zones in which the target temperatures are not hot enough or cold enough for PCR, examples of the present disclosure allow for high thermal uniformity in which all fluid in the device achieves the target temperatures. Moreover, due to the increased speed with which PCR may be completed in accordance with the present disclosure, more samples may be processed sequentially, and with less power consumption, resulting in a rapid and mobile PCR device.

In various examples of the present disclosure, a method for intermittent warming of a biologic sample includes receiving at a first end of a channel of a microfluidic device, a biologic sample including a nucleic acid, the channel disposed along a planar surface within the microfluidic device. The method includes warming a subset of a plurality of heating elements disposed adjacent to the channel of the microfluidic device to a particular temperature of a particular warming and cooling protocol, the warming and cooling protocol associated with amplification of the nucleic acid. The method further includes moving the biologic sample from the first end of the channel to a second end of the channel opposite the first end at a particular flow rate associated with the warming and cooling protocol. The method also includes intermittently warming the biologic sample using the subset of heating elements while the biologic sample moves from the first end of the channel to the second end of the channel.

Additional examples include an apparatus for intermittent warming of a biologic sample including a nucleic acid. In such examples, the apparatus includes a heatsink body, an insulating layer disposed along a planar surface within the heatsink body, and a fluidic channel disposed within the insulating layer, the fluidic channel extending from a first end of the apparatus to a second end of the apparatus opposite the first end. The apparatus further includes a plurality of heating elements arranged within the insulating layer and adjacent to the fluidic channel, each of the plurality of heating elements independently controllable to heat a biologic sample including a nucleic acid. The apparatus further includes a controller to control a temperature of the plurality of heating elements to heat and cool the biologic sample according to a particular warming and cooling protocol, the warming and cooling protocol associated with amplification of the nucleic acid.

In yet a further example, an apparatus for intermittent warming of a biologic sample including a nucleic acid comprises a heatsink body, an insulating layer disposed along a planar surface within the heatsink body, and a plurality of temperature-controlled zones. The plurality of temperature-controlled zones include a fluidic channel disposed within the insulating layer, the fluidic channel extending from a first end of the apparatus to a second end of the apparatus opposite the first end. The fluidic channel traverses from a first side of the insulating layer to a second side of the insulating layer in an alternating pattern. Each of the plurality of temperature-controlled zones include a plurality of heating elements arranged within the insulating layer and adjacent to the fluidic channel, and each of the plurality of heating elements are independently controllable to heat a biologic sample including a nucleic acid. The apparatus also includes a controller to control a temperature of the plurality of heating elements to heat and cool the biologic sample according to a particular warming and cooling protocol, the warming and cooling protocol associated with amplification of the nucleic acid.

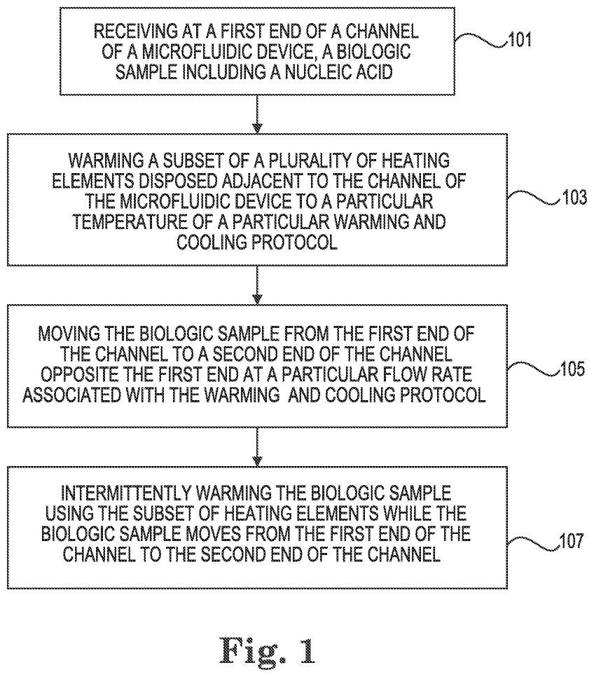

Turning now to the Figures, FIG. 1 illustrates an example method for intermittent warming of a biologic sample including a nucleic acid, consistent with the present disclosure. As illustrated in FIG. 1, the method 100 includes at 101, receiving at a first end of a channel of a microfluidic device, a biologic sample including a nucleic acid, the channel disposed along a planar surface within the microfluidic device. As used herein, a biologic sample generally refers to or includes any material containing nucleic acid, including for example, foods and allied products, clinical, and environmental samples. The biologic sample may contain any viral or cellular material, including all prokaryotic or eukaryotic cells, viruses, bacteriophages, mycoplasmas, protoplasts, and organelles. Such biological material may thus comprise all types of mammalian and non-mammalian animal cells, plant cells, algae including blue-green algae, fungi, bacteria, protozoa, etc. Representative biologic samples thus include whole blood and blood-derived products such as plasma, serum and buffy coat, urine, feces, cerebrospinal fluid or any other body fluids, tissues, cell cultures, cell suspensions, etc. In some examples, the biologic sample includes a lysate. The biologic sample may also include relatively pure starting material such as a PCR product or semi-pure preparations obtained by other nucleic acid recovery processes.

In various examples, the biologic sample may include a mixture of PCR materials. As used herein, a mixture of PCR materials refers to or includes any biological material either naturally occurring or scientifically engineered, which contains at least one nucleic acid in addition to other non-nucleic acid material, such as biomolecules (e.g., proteins, polysaccharides, lipids, low molecular weight enzyme inhibitors, oligonucleotides, primers, templates), polyacrylamide, trace metals, organic solvents, etc. Examples of naturally-occurring mixtures include, but are not limited to, whole blood, blood plasma, and other body fluids as well as tissue cell cultures obtained from humans, plants, or animals. Examples of scientifically-engineered mixtures include, but are not limited to, lysates, nucleic acid samples eluted from agarose and/or polyacrylamide gels, solutions containing multiple species of nucleic acid molecules resulting either from nucleic acid amplification methods such as PCR amplification or reverse transcription polymerase chain reaction (RT-PCR) amplification or from RNA or DNA size selection procedures, and solutions resulting from post-sequencing reactions.

Several components and reagents may be used in PCR and included in the biologic sample. Among these components are, a nucleic acid template, such as a DNA template (e.g., double-stranded DNA) that contains the target sequence to be amplified, an enzyme that polymerizes new nucleic acid strands (e.g., a polymerase enzyme such as DNA polymerase, e.g., Taq DNA polymerase), two nucleic acid primers (oligonucleotides, e.g., single-stranded) that are complementary to the 3′ (three prime) ends of each of the sense and antisense strands of the nucleic acid target, nucleoside triphosphates (NTPs) such as deoxyribonucleotide triphosphates (dNTPs) and ribonucleoside triphosphates (rNTPs), and a buffer solution providing a suitable chemical environment for amplification and optimum activity and stability of the polymerase. Specific buffer solutions may include bivalent cations, such as magnesium (Mg) or manganese (Mn) ions and monovalent cations, such as potassium (K) ions.

At 103, the method includes warming a subset of a plurality of heating elements disposed adjacent to the channel of the microfluidic device to a particular temperature of a particular warming and cooling protocol, the warming and cooling protocol associated with amplification of the nucleic acid, such as a PCR protocol. The warming and cooling of a biologic sample, as implemented by the plurality of heating elements, may be performed in accordance with a number of steps of PCR. For instance, in various examples, the subset of the plurality of heating elements is a first subset of the plurality of heating elements and the particular temperature is a first temperature, such as a denaturing temperature. In such examples, the method further includes warming a second subset of the plurality of heating elements disposed adjacent to the channel of the microfluidic device to a second temperature of the particular warming and cooling protocol, such as an annealing temperature.

PCR is carried out in a reaction volume and is performed in three distinct phases. The biologic sample contains one nucleic acid to be amplified, which is termed “the original” or “sense” strand. In the biologic sample, the sense strand can be in a double-strand form with its complementary strand, which is termed “the complement” or “antisense” strand. If the sense and antisense strands are present as a double-strand DNA molecule, this double-strand DNA molecule is denatured in a first step of PCR, i.e., the double-strand DNA molecule is split into two single strands, e.g., the sense and antisense strands.

In the first step of PCR, the two strands of a double-stranded molecule (e.g., DNA or RNA) are physically separated at a high temperature in a process called denaturation or melting. Denaturation occurs at a temperature, which is termed the denaturing temperature. The biologic sample further contains at least two primers. Primers refer to or include short single-strand nucleic acid segments, which are also known as oligonucleotides, which are a complementary sequence to the target nucleic acid sequence. One of the primers is termed a forward primer while the other is termed a reverse primer. The forward primer is complementary to the 3′-end of the sense strand. The reverse primer is complementary to the 3′-end of the antisense strand.

In the second step of PCR, the temperature is lowered, and the primers hybridize or bind to their complementary sequences on the nucleic acid sequence. The two, now double-stranded nucleic acid strands then become templates for an enzymatic reaction using a polymerase to transcribe (e.g., replicate, synthesize, or assemble) a new nucleic acid strand from free nucleotides that are also found in the reaction volume. The forward primer hybridizes to a sequence in the sense strand while the reverse primer hybridizes to a sequence in the antisense strand. The hybridization of the primers with the complementary sequences of the sense or antisense strand is termed annealing. This second step takes place at a temperature termed the annealing temperature.

The biologic sample further contains a DNA polymerase. In a third step, the DNA polymerase synthesizes a copy of the complement starting from the forward primer and synthesizes a copy of the sense strand starting from the 5′ end of the reverse primer. Throughout the synthesis, the copy of the antisense strand also hybridizes with the sense strand and the copy of the sense strand hybridizes with the antisense strand. This third step is termed elongation and is carried out at a temperature called the elongation temperature.

After the elongation step, the first, second, and third steps are repeated until the desired extent of amplification is achieved, wherein multiple copies of the sense and antisense strands are made. As PCR progresses, the nucleic acid generated is itself used as a template for replication, setting in motion a chain reaction in which the original nucleic acid template is exponentially amplified. Each of the respective temperatures for denaturation, annealing, and elongation may be achieved using the heating elements as described herein.

The denaturing temperature may be chosen such that the single strands of the nucleic acid denature while not effecting, e.g., damaging, the polymerase. In some examples, the denaturing temperature is about 94 degrees C. In some examples, the annealing temperature depends on the sequence and length of the primers. For instance, the primers may be designed for an annealing temperature between about 50 degrees C. and 65 degrees C. Additionally, the elongation temperature may depend on the DNA polymerase used. For example, if using Taq DNA polymerase, an elongation temperature of about 72 degrees C. may be used. After elongation, the temperature is returned to 94 degrees C. for denaturation of the double-stranded DNA to single-stranded DNA. This cycling from denaturation-annealing-elongation is repeated a number of times, typically 20 to 40 cycles.

In accordance with the warming and cooling protocol discussed above, in various examples, the method may include raising and lowering the temperature of the biologic sample using the plurality of heating elements and according to the particular warming and cooling protocol. For instance, various heating elements may be used to reach a particular denaturation temperature, a particular annealing temperature, and/or a particular elongation temperature. In some examples, the method may further include intermittently cooling, according to the particular warming and cooling protocol, the biologic sample using a cooling agent flowing through a plurality of cooling chambers disposed adjacent to the channel, as discussed further herein.

The plurality of heating elements may be arranged and heated so as to form various temperature-controlled zones separated by insulative zones, also referred to as adiabatic zones. As used herein, temperature-controlled zones refer to or include both heating and cooling zones. For instance, in some examples, the subset of the plurality of heating elements are a first subset of the plurality of heating elements and the particular temperature is a first temperature (e.g. temperature-controlled zones). In such examples, the method includes warming a second subset of the plurality of heating elements to a second temperature (e.g., temperature-controlled zones of a different temperature) of the particular warming and cooling protocol. The method may further include not warming (e.g., cooling zones) a third subset of the plurality of heating elements. In such examples, the third subset of the plurality of heating elements are associated with cooling zones of the microfluidic device. In some examples, the method includes digitally controlling the temperature of the plurality of heating elements, so as to form the zones described herein.

In some examples, the microfluidic device includes a first chamber including a plurality of heating elements and a second chamber including a plurality of heating elements. In such examples, intermittently warming the biologic sample using the subset of heating elements includes cycling the biologic sample between the first chamber and the second chamber a specified number of times according to the warming and cooling protocol, as discussed further herein.

At 105, the method further includes moving the biologic sample from the first end of the channel to a second end of the channel opposite the first end at a particular flow rate associated with the warming and cooling protocol. In various examples, the rate of fluid flow through the channel may be adjusted, and/or the temperatures of the heating elements may be modified according to a particular warming and cooling protocol.

At 107, the method also includes intermittently warming the biologic sample using the subset of heating elements while the biologic sample moves from the first end of the channel to the second end of the channel. As the biologic sample moves through the channel of the microfluidic device, the heating elements change the temperature of the biologic sample according to the warming and cooling protocol. For instance, the heating elements heat the biologic sample in accordance with the denaturation, annealing, and elongation process described above, as the biologic sample progresses through the channel.

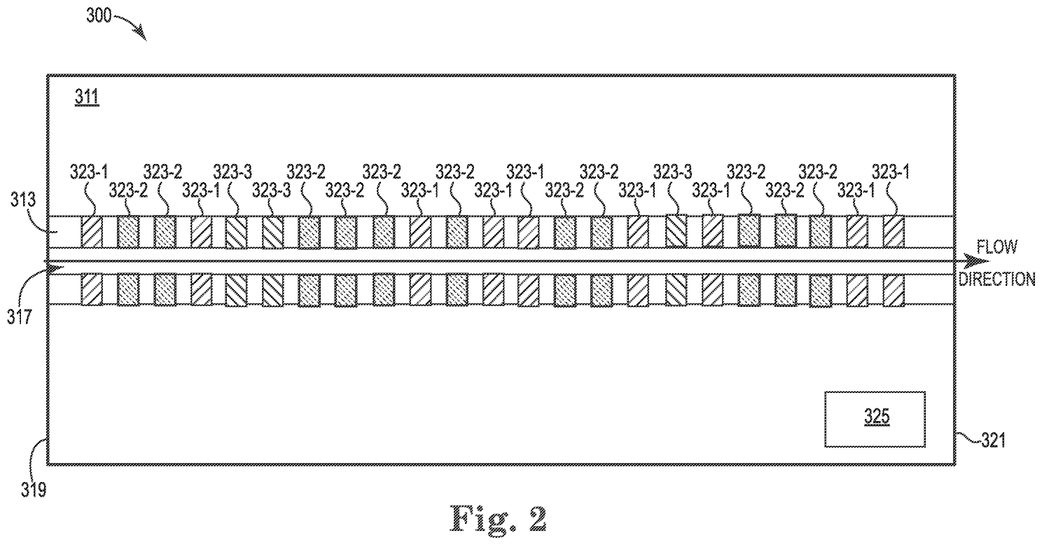

FIG. 2 illustrates an example apparatus including a plurality of heating elements, consistent with the present disclosure. Consistent with the present disclosure, heating elements allow for customized, and modifiable, thermal protocols implemented by a PCR device. The protocol can be changed by means of a computer which turns on and off different temperature-controlled zones, without mechanical changes to the PCR device.

In various examples, temperature-controlled zones may be implemented by heating elements, as illustrated in FIG. 2. As used herein, a heating element refers to or includes circuitry to apply heat at a specified temperature to the surrounding structures, including the biologic sample, in the apparatus. As illustrated in FIG. 2, an apparatus 300 for intermittent warming of a biologic sample including a nucleic acid includes a heatsink body 311, an insulating layer 313 disposed along a planar surface 315 within the heatsink body 311, and a fluidic channel 317 disposed within the insulating layer 313. As used herein, a heatsink body refers to or includes a substance with a high conductivity and heat capacity. Non-limiting examples of a substance for a heatsink body may include copper, aluminum, iron, silicon, and combinations thereof. Also as used herein, an insulating layer refers to or includes an insulating substance. Non-limiting examples of a substance for an insulating layer may include plastic, adhesive, polymers such as Su8, oxides, and combinations thereof. The fluidic channel 317 extends from a first end 319 of the apparatus to a second end 321 of the apparatus 300 opposite the first end 319. The fluidic channel may receive the biologic sample described herein.

In various examples, a plurality of heating elements 323 may be arranged within the insulating layer 313 and adjacent to the fluidic channel 317. For the ease of illustration, each of the heating elements 323 are not numbered in FIG. 2. However, the heating elements 323 may be arranged around the fluidic channel 317. Each of the plurality of heating elements 323 may be independently controllable and monitored to warm a biologic sample including a nucleic acid, as described herein. For instance, heating elements 323-1 may be set at a high temperature, such as a denaturation temperature for example. As illustrated in FIG. 2, several of the heating elements 323 may be set to this high temperature and are therefore labeled as heating elements 323-1. Similarly, heating elements 323-2 may be set at a warmed temperature that is different than that of heating elements 323-1, such as an elongation temperature for example. Several of the heating elements 323 may be set at this warmed temperature and are therefore labeled as heating elements 323-2. Moreover, heating elements 323-3 may be turned off and act as a heat sink, drawing heat away from the fluidic channel 317, thereby bringing the biologic sample to the anneal temperature. Several of the heating elements 323 may be turned off and are therefore labeled as heating elements 323-3. While the heating elements 323 on the opposing side of the fluidic channel 317 are not labeled in FIG. 2, FIG. 2 illustrates that the heating elements 323 on the opposing side of the fluidic channel 317 have a same temperature setting.

Additionally, the apparatus 300 may include a controller 325 to control and monitor a temperature of the plurality of heating elements 323 to warm and cool the biologic sample according to a particular warming and cooling protocol, the warming and cooling protocol associated with amplification of the nucleic acid. For instance, the controller 325 may control the temperature at which each of the heating elements 323 is set. The controller 325 may set the temperature of each of the heating elements 323 in a particular pattern for nucleic acid amplification. The apparatus 300 may further include temperature sensor resistors (not illustrated in FIG. 2) to sense the temperature of the biologic sample. The temperature readings obtained by the temperature sensor resistors may be collected by the controller 325 and used to set the temperature of the heating elements 323. As an example, each heating element may include a thin film of circuitry deposited on a silicon substrate. The thin film may include resistor circuitry for controlling the temperature of the heating element, as well as temperature sense circuitry for measuring.

The biologic sample may be warmed by different zones within the apparatus 300. Accordingly, the controller 325 may set the temperature of the plurality of heating elements 323 in a pattern of temperature-controlled zones, and cooling zones. For example, heating elements 323-1 may be associated with a temperature-controlled zone, heating elements 323-2 may be associated with a cooling zone, and heating elements 323-3 may be associated with a temperature-controlled zone of a different temperature than heating elements 323-1.

As illustrated in FIG. 2, the plurality of heating elements 323 are arranged on opposing sides of the fluidic channel 317 within the insulating layer 313. Each heating element may be independently controlled by the controller 325. For instance, the controller 325 may form a temperature-controlled zone by warming opposing heating elements, e.g., the corresponding heating elements on opposite sides of fluidic channel 317. Additionally and/or alternatively, the controller 325 may form a temperature-controlled zone by warming one heating element of a pair of opposing heating elements 323.

As each of the plurality of heating elements 323 is independently controllable, zones of different temperature may be achieved in apparatus 300 without mechanical changes. For instance, each of heating elements 323-2 may be turned off, such that heat dissipates into the heatsink body. Due to heat dissipating from the fluidic channel 317 into the heatsink body 311, the temperature of the biologic sample in the fluidic channel 317 lowers to an ambient temperature (such as about 25 C). In contrast, each of heating elements 323-1 may be set to 100 C. Heating elements 323-2 may be set to an intermediate temperature, such as 85 C. In such examples, a temperature-controlled zone is defined by at least one heating element set to a temperature warmer than room temperature, and a cooling zone is defined by at least one heating element that is turned off, as discussed herein. As such, the apparatus 300 may include a plurality of cooling zones disposed between alternating temperature controlled zones, each of the plurality of cooling zones including a plurality of heating elements arranged within the insulating layer and adjacent to the fluidic channel, each of the plurality of heating elements independently controllable to heat a biologic sample including a nucleic acid.

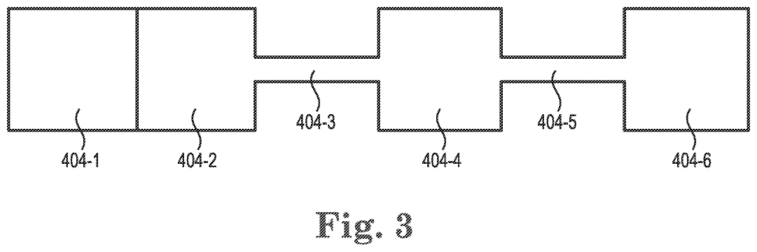

FIG. 3 illustrates an example apparatus including narrowed adiabatic zones, consistent with the present disclosure. In various examples, the shape and/or diameter of the fluidic channel may be modified in order to reduce the amount of time it takes to change the temperature of the biologic sample. The example apparatus 400 includes narrowed adiabatic zones to facilitate rapid heating and cooling. Particularly, FIG. 3 illustrates a cross-section of the fluidic channel without the remainder of the apparatus, such as the heating elements. In the example illustrated in FIG. 3, the fluidic channel 417 includes a plurality of adiabatic zones 404-1, 404-3, and 404-5. The diameter of the adiabatic zones 404-3 and 404-5 may be reduced relative to a diameter of a remainder of the fluidic channel, such as zones 404-2, 404-4, and 404-6. Zones 404-2 and 404-6 may have heating elements disposed on opposing sides of the fluidic channel 417 that apply heat of 100 C. Similarly, zone 404-4 may have heating elements disposed on opposing sides of the fluidic channel 417 that apply heat of 0 C. By disposing the narrowed adiabatic zones 404-3 and 404-5 between zones of high heat and low heat, the temperature of the biologic sample within zones 404-2, 404-4, and 404-6 may be more accurately controlled.

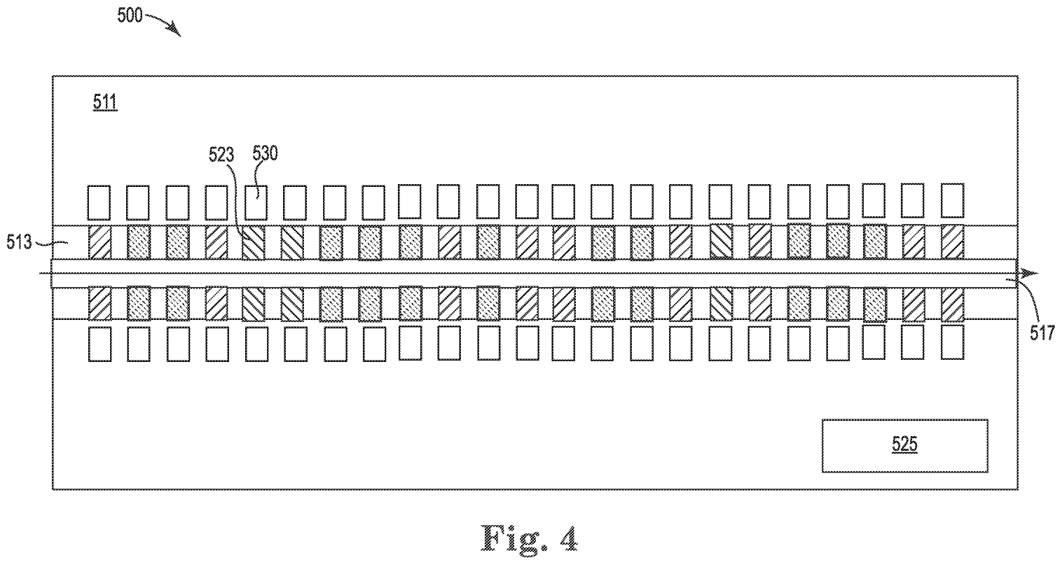

FIG. 4 illustrates an example apparatus including liquid cooling elements, consistent with the present disclosure. In various examples, a liquid cooling agent may pass over select portions of the heating elements to further absorb heat and rapidly reduce the temperature of the biologic sample in the fluidic channel 517. For instance, as illustrated in FIG. 4, the apparatus 500 may include a plurality of liquid cooling elements 530 disposed within the heatsink body 511 and adjacent to the plurality of heating elements 523, each of the plurality of liquid cooling elements 530 to selectively pass a liquid cooling agent along a plane orthogonal to a direction of the flow of the biologic sample. For instance, the liquid cooling agent may selectively pass through different ones of the liquid cooling elements 530, along the Z axis. In various examples, the liquid cooling agent refers to or includes a liquid with a high heat capacity which does not corrode the heatsink body 511 or other components of the apparatus 500. As a non-limiting example, the liquid cooling agent may include water, antifreeze, oil, hydraulic fluid, water with anti-bacterial additives, a Fluorinert™ such as FC-72, a refrigerant, or combinations thereof.

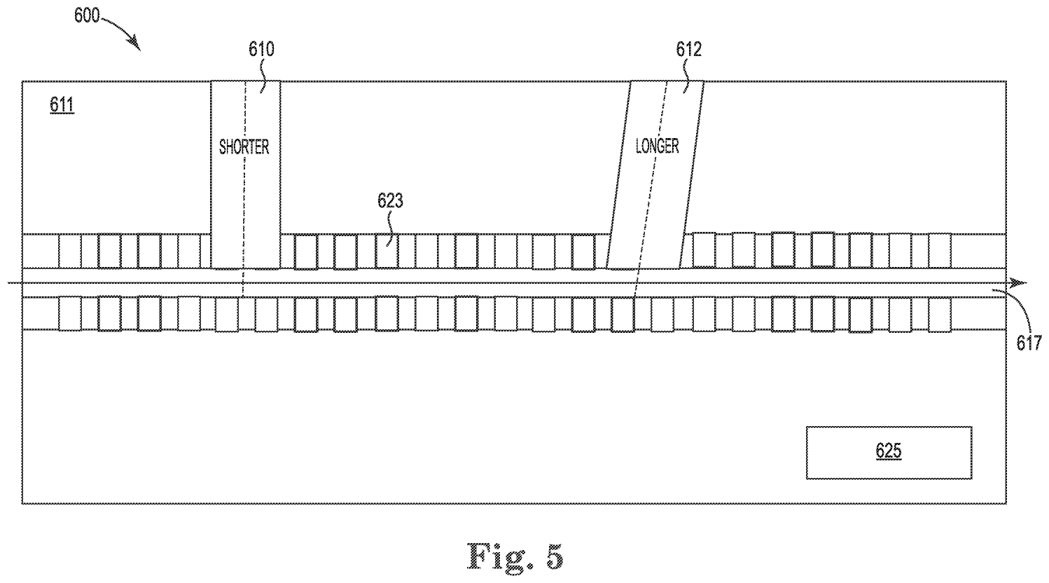

FIG. 5 illustrates an example apparatus including multiple viewing windows, consistent with the present disclosure. In various examples, it may be beneficial to view the progress of PCR, such as may be implemented during Real-Time PCR. To facilitate viewing of amplification progress, the apparatus may include transparent viewing windows. For instance, apparatus 600 may include a transparent viewing window (410 and 412) traversing a width of the heatsink body 611 to the fluidic channel 617. In some examples, the transparent viewing window may orthogonally traverse the heatsink body 611, such as illustrated by viewing window 410. In other examples, the transparent viewing window may traverse the heatsink body 611 at an angle, such as illustrated by viewing window 410. By angling the viewing window (e.g., 410) a larger cross section of the fluidic channel 617 can be viewed, and greater data may be obtained with regard to the progress of nucleic acid amplification.

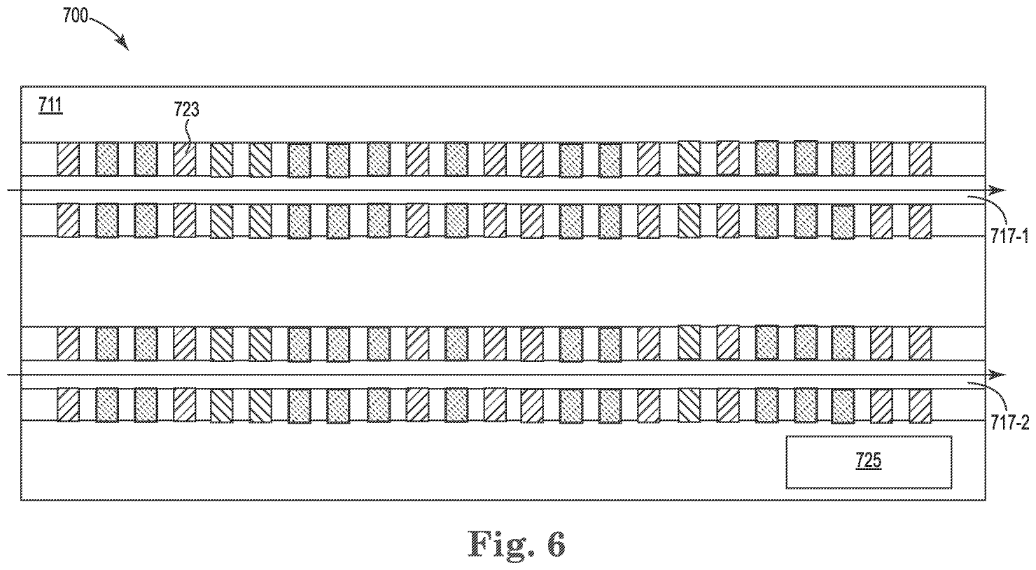

FIG. 6 illustrates an example apparatus 700 including a multiplexed fluidic channel, consistent with the present disclosure. To process multiple samples at one time, a plurality of fluidic channels may be stacked upon one another. For instance, fluidic channel 717-1 and fluidic channel 717-2 may be disposed within a same heatsink 717. While FIG. 6 illustrates two fluidic channels in apparatus 700, examples are not so limited and apparatus 700 may have any number of fluidic channels stacked upon one another to form a multiplexed fluidic device.

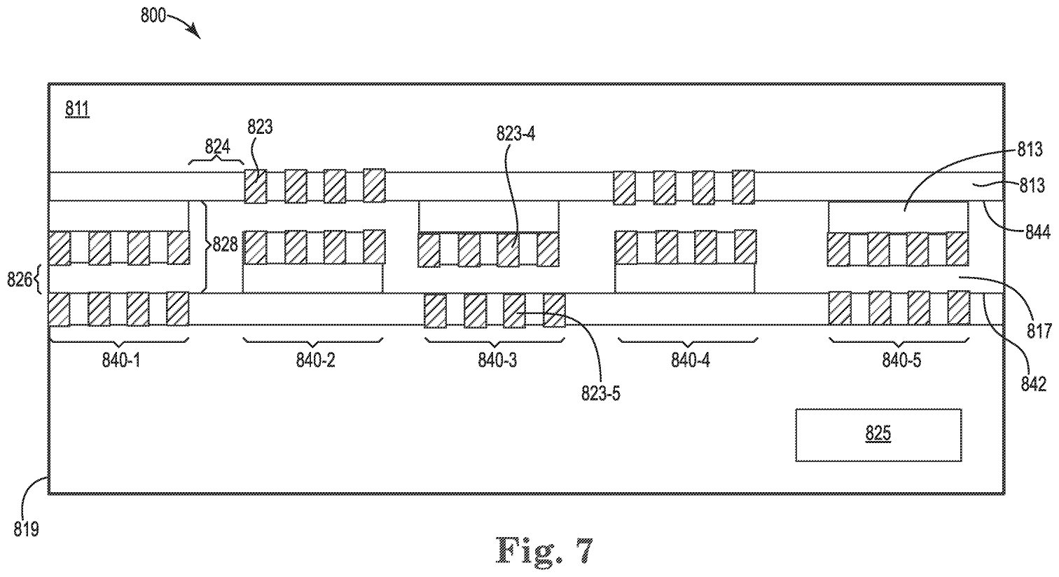

FIG. 7 illustrates an example apparatus including a non-straight fluidic channel, consistent with the present disclosure. In various examples, including a non-straight fluid channel changes the fluid fields within zones such that target temperatures are achieved more rapidly. Referring to FIG. 7, an apparatus 800 for intermittent warming of a biologic sample including a nucleic acid includes a heatsink body 811, an insulating layer 813 disposed along a planar surface within the heatsink body 811, and a plurality of temperature controlled zones 840-1, 840-2, 840-3, 840-4, and 840-5 (referred to collectively as temperature controlled zones 840). For instance, the insulating layer 813, as illustrated, is disposed along the x axis. Each of the temperature-controlled zones includes a fluidic channel 817 disposed within the insulating layer 813, the fluidic channel 817 extending from a first end 819 of the apparatus to a second end 821 of the apparatus 800 opposite the first end 819.

In various examples, each of the plurality of temperature-controlled zones 840 includes a plurality of heating elements 823 arranged within the insulating layer 813 and adjacent to the fluidic channel 817. As discussed herein, each of the plurality of heating elements 823 may be independently controllable to heat a biologic sample including a nucleic acid. Moreover, the fluidic channel 817 may traverse from a first side 842 of the insulating layer 813 to a second side 844 of the insulating layer 813 in an alternating pattern, as illustrated. For instance, after the termination of temperature-controlled zone 840-1, the fluidic channel 817 may traverse along the y axis toward the second side 844 to the beginning of temperature-controlled zone 840-2. After the termination of temperature-controlled zone 840-2, the fluidic channel 817 may traverse along the y axis toward the first side 842 to the beginning of temperature-controlled zone 840-3, and so forth.

As illustrated in FIG. 7, the fluidic channel 817 may be constricted where the heating elements are disposed (e.g., heating elements 823), and the fluidic channel 817 may be widened between temperature-controlled zones (e.g., where the fluidic channel 817 is insulated). As a non-limiting example, the width 826 of the fluidic channel 817 in a temperature controlled zone such as 840-1 may be between about 10 microns to about 100 microns, whereas the width 828 of the fluidic channel 817 in the adiabatic zone between the temperature controlled zones may be about two to five times the width 826 in the temperature controlled zone.

The distance between temperature-controlled zones, such as distance 824 between temperature-controlled zone 840-1 and 840-2 may vary. For instance, in some examples the distance 824 between temperature-controlled zones may be approximately 50 microns

While various examples are described herein, in which heating elements 823 on opposite sides of fluidic channel 817 are set to a same temperature, examples are not so limited. For example, heating element 823-4 and heating element 823-5 may be set to a same temperature, or heating element 823-4 and heating element 823-5 may be set to different temperatures.

In additional examples, the apparatus 800 may include a controller 825 to control a temperature of the plurality of heating elements 823 to warm and cool the biologic sample according to a particular warming and cooling protocol, the warming and cooling protocol associated with amplification of the nucleic acid.

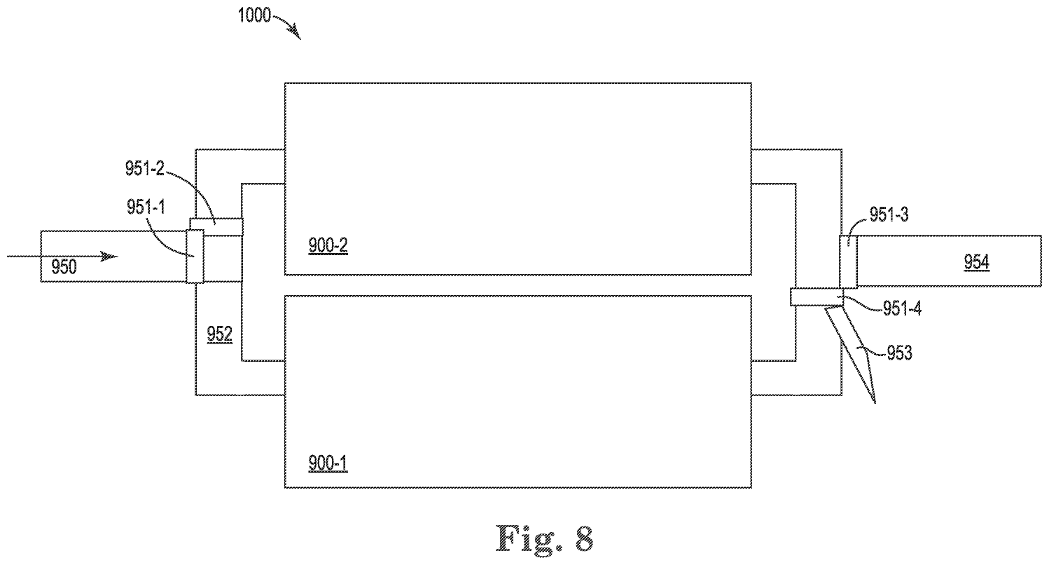

FIG. 8 illustrates an example apparatus 1000 including a looped fluidic channel, consistent with the present disclosure. In various examples, a plurality of fluidic channels may be coupled together in a loop, so as to reduce the length of the channel and reduce manufacturing costs. For instance, as the biologic fluid enters channel 950, a series of valves 951-1, 951-2, 951-3, and 951-4 may control the flow of the biologic fluid through apparatus 900-1 and apparatus 900-2. Each of apparatus 900-1 and apparatus 900-2 may be similar to any one of the apparatuses illustrated in FIGS. 1-7. As the fluid enters channel 950, valve 951-1 may open to allow fluid to enter channel 952. Valve 951-2 may shut to ensure that the biologic fluid flows towards apparatus 900-1. Once the biologic fluid has entered channel 952, valve 951-1 may shut.

Channel 952 may direct the biologic fluid to apparatus 900-1 and through apparatus 900-1 as described herein. For example, referring to FIG. 7, channel 952 may be coupled to fluidic channel 817, such that fluid enters the fluidic channel from channel 952. Once the biologic fluid passes through apparatus 900-1, valve 951-4 may open to permit the biologic fluid to pass to apparatus 900-2. Valve 951-3 may remain closed to ensure the biologic fluid passes to apparatus 900-2. Once the biologic fluid passes to apparatus 900-2, valve 951-4 may shut.

Channel 952 may direct the biologic fluid to apparatus 900-2 and through apparatus 900-2 as described herein. For example, referring to FIG. 7, channel 952 may be coupled to fluidic channel 817, such that fluid enters the fluidic channel from channel 952. Once the biologic fluid passes through apparatus 900-2, valve 951-2 may open to permit the biologic fluid to pass to apparatus 900-1 again. Valve 951-1 may remain closed to ensure the biologic fluid passes to apparatus 900-1. Once the biologic fluid passes to apparatus 900-1, valve 951-2 may shut again.

The biologic fluid may circulate between apparatus 900-1 and apparatus 900-2 several times to repeat the nucleic acid amplification process. Once target amplification is achieved, the biologic fluid may be released. For instance, as the biologic fluid passes through valve 951-4, valve 951-3 may be opened to allow the biologic fluid to enter channel 954 and exit apparatus 1000.

In various examples, each of valves 951-1, 951-2, 951-3, and 951-4 may be individually actuated by circuitry (not illustrated in FIG. 8) coupled to the valves 951-1, 951-2, 951-3, and 951-4. Additionally and/or alternatively, the circuitry may automatically open and shut in a controlled manner consistent with the heating and cooling protocol. Moreover, in various examples the apparatus 1000 includes an inlet 953. After the reaction has gone to completion, clean fluid may be injected from 953 while valves 951-4 and 951-1 are closed and 951-2 and 951-3 are open. This will flush all the reacted fluid out of channel 954 where the sample may be collected and analyzed.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

1. A method, comprising:
receiving at a first end of a channel of a microfluidic device, a biologic sample including a nucleic acid, the channel disposed along a planar surface within the microfluidic device;
warming a subset of a plurality of heating elements disposed adjacent to the channel of the microfluidic device to a particular temperature of a particular warming and cooling protocol, the warming and cooling protocol associated with amplification of the nucleic acid;
moving the biologic sample from the first end of the channel to a second end of the channel opposite the first end at a particular flow rate associated with the warming and cooling protocol; and
intermittently warming the biologic sample using the subset of heating elements while the biologic sample moves from the first end of the channel to the second end of the channel.

2. The method of claim 1, wherein the subset of the plurality of heating elements is a first subset of the plurality of heating elements and the particular temperature is a first temperature, the method further including:
warming a second subset of the plurality of heating elements disposed adjacent to the channel of the microfluidic device to a second temperature of the particular warming and cooling protocol.

3. The method of claim 1, further including intermittently cooling, according to the particular warming and cooling protocol, the biologic sample using a cooling agent flowing through a plurality of cooling chambers disposed adjacent to the channel.

4. The method of claim 1, wherein the subset of the plurality of heating elements are a first subset of the plurality of heating elements and the particular temperature is a first temperature, the method including:
warming a second subset of the plurality of heating elements to a second temperature of a particular warming and cooling protocol; and
not warming a third subset of the plurality of heating elements, the third subset of the plurality of heating elements associated with cooling zones of the microfluidic device.

5. The method of claim 4, including digitally controlling and monitoring the temperature of the plurality of heating elements.

6. The method of claim 1, wherein the microfluidic device includes a first chamber including a plurality of heating elements and a second chamber including a plurality of heating elements, and wherein intermittently warming the biologic sample using the subset of heating elements includes cycling the biologic sample between the first chamber and the second chamber a specified number of times according to the warming and cooling protocol.

7. An apparatus, comprising:
a heatsink body;
an insulating layer disposed along a planar surface within the heatsink body;
a fluidic channel disposed within the insulating layer, the fluidic channel extending from a first end of the apparatus to a second end of the apparatus opposite the first end;
a plurality of heating elements arranged within the insulating layer and adjacent to the fluidic channel, each of the plurality of heating elements independently controllable to heat a biologic sample including a nucleic acid; and
a controller to control a temperature of the plurality of heating elements to heat and cool the biologic sample according to a particular warming and cooling protocol, the warming and cooling protocol associated with amplification of the nucleic acid.

8. The apparatus of claim 7, wherein the controller is to set the temperature of the plurality of heating elements in a pattern of temperature-controlled zones, and cooling zones.

9. The apparatus of claim 7, wherein the fluidic channel includes a plurality of adiabatic zones, a diameter of each of the adiabatic zones reduced relative to a diameter of a remainder of the fluidic channel.

10. The apparatus of claim 7, further including a plurality of liquid cooling elements disposed within the heatsink body and adjacent to the plurality of heating elements, each of the plurality of liquid cooling elements to selectively pass a liquid cooling agent along a plane orthogonal to a direction of a flow of the biologic sample.

11. The apparatus of claim 7, further including a transparent viewing window traversing a width of the heatsink body to the fluidic channel.

12. An apparatus, comprising:
a heatsink body;
an insulating layer disposed along a planar surface within the heatsink body;
a plurality of temperature-controlled zones, including:
a fluidic channel disposed within the insulating layer, the fluidic channel extending from a first end of the apparatus to a second end of the apparatus opposite the first end, wherein the fluidic channel traverses from a first side of the insulating layer to a second side of the insulating layer in an alternating pattern;
each of the plurality of temperature-controlled zones including a plurality of heating elements arranged within the insulating layer and adjacent to the fluidic channel, each of the plurality of heating elements independently controllable to heat a biologic sample including a nucleic acid; and
a controller to control a temperature of the plurality of heating elements to heat and cool the biologic sample according to a particular warming and cooling protocol, the warming and cooling protocol associated with amplification of the nucleic acid.

13. The apparatus of claim 12, further including a plurality of cooling zones disposed between alternating temperature controlled zones, each of the plurality of cooling zones including a plurality of heating elements arranged within the insulating layer and adjacent to the fluidic channel, each of the plurality of heating elements independently controllable to heat a biologic sample including a nucleic acid.

14. The apparatus of claim 12, wherein the plurality of heating elements are arranged on opposing sides of the fluidic channel within the insulating layer, the controller to form a temperature-controlled zone by warming opposing heating elements.

15. The apparatus of claim 12, wherein the plurality of heating elements are arranged on opposing sides of the fluidic channel within the insulating layer, the controller to form a temperature-controlled zone by warming one heating element of a pair of opposing heating elements.
